(19) United States
(12) Patent Application Publication
(10) Pub. No.: US 2011/0015571 A1
(43) Pub. Date: Jan. 20, 2011

(54) Balloon Catheter

(75) Inventors: Jokum Christian Voss, Espergaerde (DK); Pernille Merethe Guldager, Kgs. Lyngby (DK)

Correspondence Address: COLOPLAST A/S;Attention: Corporate Patents, Holtedam 1, DK-3050 Humlebaek, DK

(73) Assignee: COLOPLAST A/S, Humlebaek (DK)

(21) Appl. No.: 12/741,595

(22) PCT Filed: Nov. 6, 2008
(86) PCT No.: PCT/DK08/50269

§ 371 Date: Sep. 13, 2010

(30) Foreign Application Priority Data
Nov. 6, 2007 (DK) PA 2007 01571

(51) Int. Cl. A61M 29/00

(52) U.S. Cl. 604/103

(57) ABSTRACT

The present invention relates to the attachment of balloon walls to catheters in order to provide a balloon catheter. In particular it relates to adhering the balloon walls to the catheters in order to optimize an even inflation of the balloon.

### FIELD OF THE INVENTION

The present invention relates to the attachment of balloon walls to catheters in order to provide a balloon catheter. In particular it relates to adhering the balloon walls to the catheters in order to optimize an even inflation of the balloon.

### BACKGROUND

Balloon catheters are used for many different medical applications in order to retain a catheter within a lumen of the human body.

One type of use is anal irrigation, wherein a fluid is ejected into the rectum and lower bowels of a person for example in order to loosen constipation, stimulate the peristaltic, introduce contrast enhancement fluid, collect feces or introduce a medicament.

It is, especially for smaller balloon catheter e.g. used for kids, important that the balloon inflates evenly so that the catheter does not lies against the wall of the rectum with the balloon ballooning out to one side. This may compromise the seal of the inflated balloon and/or cause discomfort to the user and maybe even scratch the rectum wall as the catheter typically is stiffer than the inflated balloon.

It has shown that in order to provide an even inflation of the balloon many factors come into play. One such factor is the attachment of the balloon wall to the catheter body.

In commonly known rectal catheters of the balloon type, the balloon wall is typically joined to the catheter body at two connection zones distributed along the longitudinal extent (length) of the catheter, where the balloon wall between the connection zones is the part which is inflated.

Such joining may e.g. be provided by an adhesive which is applied between the balloon wall and the catheter body in the connection zones. In other embodiment welding provides joining or alternatively using a solvent will partly dissolve the balloon wall and catheter body wherein the material fuses together in a joint.

The adhesive is applied on the catheter body in an annular ring where after the balloon wall is applied. As the balloon wall presses against the catheter body the adhesive will be pressed out to the sides, i.e. in a longitudinal direction, in random patterns. This results in the longitudinal distance in which gas or liquid may be injected into the balloon in order to inflate it varies considerably around the catheter. In a typical rectal catheter having a standard size for use with adults such distance may vary with several millimeters, even up and above one centimeter.

It has shown that these uneven distributions, in particular along the inner edge of the adhesive which is closest to the other connection zone, in some cases have an effect on the initial inflation of the balloon and/or on the final shape of the balloon.

Thus, by being able to control how excess adhesive flows it is possible to provide a more evenly inflated balloon.

In a similar manner when using welding the melted excess welding material, e.g. from the filler or the catheter body material itself, can be led down the outflow surface away from the balloon wall.

Even further, when using solvents to join the balloon wall and catheter body in the connection zone, excess solvent may likewise be led down the outflow surface.

### SUMMARY OF THE INVENTION

The current invention relates to a balloon catheter comprising an elongated catheter body extending along a longitudinal axis, where a balloon wall is coupled to the catheter body in at least two annular coupling zones, wherein in at least one of the coupling zones the balloon wall is joined to the catheter body by a joining material, said joining material being provided on a first joining surface of a rim arranged annularly, with a first radius, around the longitudinal axis and extending along the longitudinal axis; and where the joining surface, in a direction towards the opposite coupling zone, continues into an outflow surface which form an angle to the first joining surface, said outflow surface extending from the first radius toward a decreased radius.

This provides a route for excess joining material to escape down the outflow surface so that it does not join to the balloon wall in undesired areas.

As may be understood from the above the joining material may differ depending on which process is used to joining the balloon wall and catheter body in the connection zone. Accordingly, the joining material may be an adhesive if the elements are adhered together; a filler or surplus catheter body material if they are welded together; or a solvent if they are to be partly dissolved in order to be joined together.

Within this text the term ‘rim’ should be understood as being an annular part, around the longitudinal axis, of the catheter; for example in the form of an annular band. The rim having a surface suited to be used as a surface to be joined with another element to, e.g. the balloon wall.

In order to reduce the risk that excess joining material spills over from the connection zone into a direction towards the opposite coupling zone guiding means for guiding the joining material in a direction away from the opposite coupling zone are provided on at least one of the coupling zones.

One such guiding means could comprise an elevated edge provide on at least one joining surface proximal to the opposite coupling zone.

The guiding means may also comprise at least one rib provided on the at least one joining surface. This may be used to control the speed on which the joining material flows by using the rib(s) as barriers.

By providing a large angle the outflow surface allows joining material to be removed quicker away from the balloon wall. Thus, the angle that the outflow surface forms to the joining surface may be above 20°, such that the joining surface tapers from the first radius towards a decreased radius in a direction towards the opposite coupling zone.

As mentioned above, the joining material may be an adhesive. The viscosity of the adhesive also has an effect on how the adhesive is lead away and the type and size of undesired adhesive areas. The viscosity of adhesives typically used for this application will be below 10.000 cP.

In order to provide even more control of the adhesive (joining material) and how it flows, the first joining surface may be planar.

In one embodiment the first joining surface is parallel to the longitudinal axis. This provides an even flow of the adhesive (joining material) to both sides.

Alternatively the first joining surface forms an angle with the longitudinal axis whereby the surface tapers inwards in the longitudinal direction away from the opposite coupling zone. This functions as guiding means and allows the excess adhesive (joining material) to flow away.

In another embodiment a first annular reservoir groove is provided on at least one side of the first joining surface. This provides a reservoir for collecting excess adhesive (joining material).

In yet another embodiment a second joining surface is in the longitudinal direction provided on the opposite side of the first annular reservoir groove relative to the first second joining surface.

The catheter body may be injection moulded. This process allows for well-defined surfaces and edges on catheter bodies, which are suitable in the present invention.

### BRIEF DESCRIPTION OF THE DRAWINGS

FIG. 1 shows in section a part of a balloon catheter as known in the art,

FIG. 2 shows in section a part of a first embodiment of a balloon catheter according to the invention,

FIG. 3 shows in section a part of a second embodiment of a balloon catheter according to the invention,

FIG. 4 shows in section a part of a third embodiment of a balloon catheter according to the invention,

FIG. 5 shows in section a part of a fourth embodiment of a balloon catheter according to the invention, and

FIG. 6 shows in section a part of a fifth embodiment of a balloon catheter according to the invention.

### DETAILED DESCRIPTION

In the following, unless otherwise specified, the term proximal refers to a part which is closer to the human body when the insertion device is about to be inserted relative to a corresponding other distal part.

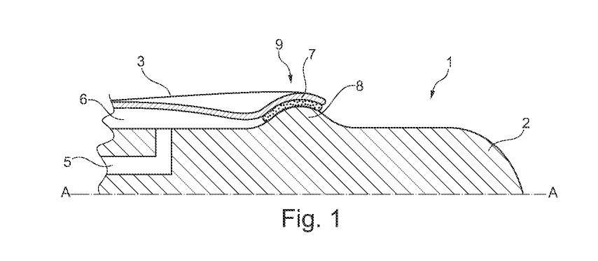

The proximal part of a commonly known rectal balloon catheter 1 is shown in part in FIG. 1. The balloon catheter is formed of a catheter body 2 and a balloon wall 3 joined to the catheter body in a connection zone 4.

An inflation channel 5 communicates with the inflation lumen 6 into which an inflation medium (e.g. a gas or liquid) is pumped in order to expand the balloon wall in a radial direction relative to the longitudinal axis A-A of the balloon catheter.

The balloon wall is by an adhesive 7 joined to the catheter body in the connection zone. The adhesive is applied to the surface of an annular rib 8. As the balloon wall is pressed against the catheter body in order for the adhesive to adhere the adhesive begin to flow outwardly (i.e. in a longitudinal direction). As shown in the section the adhesive will in some areas flow longer along both the balloon wall and the catheter body compared to other areas. This results in an uneven distribution of adhesive in the longitudinal direction, which as mentioned above, have an effect on the initial inflation of the balloon wall and on the final inflated shape of the balloon wall.

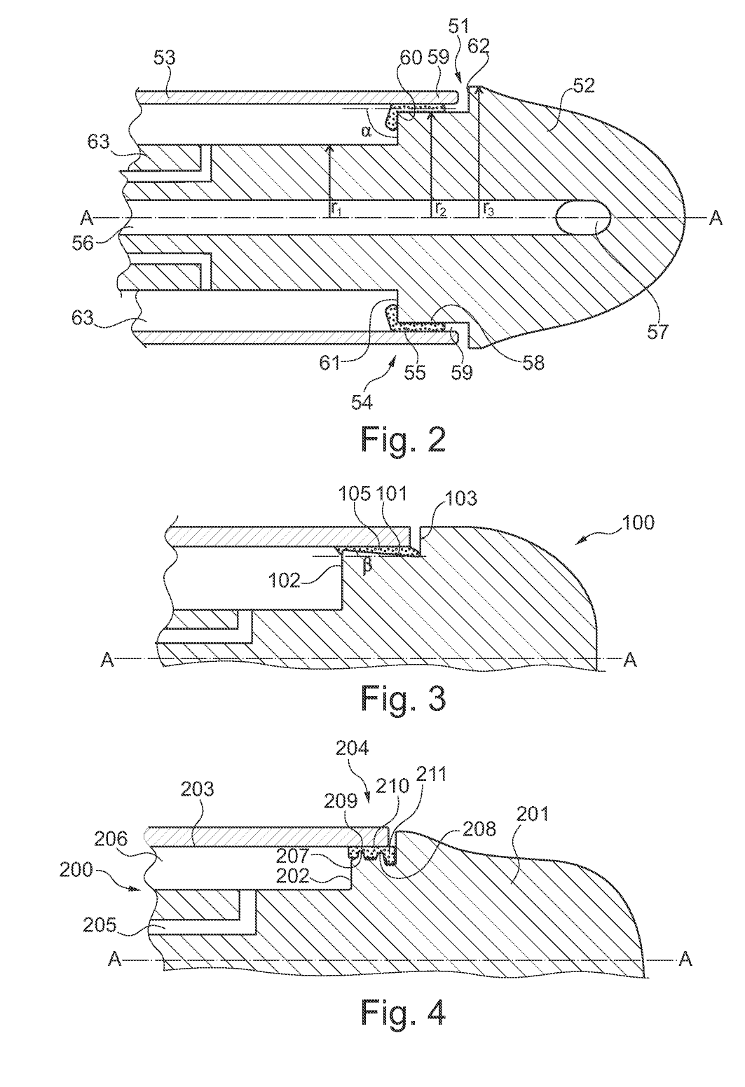

The proximal section of one embodiment of a balloon catheter 51 is shown in FIG. 2. The balloon catheter extends longitudinal along the axis A-A. The distal end comprises a catheter body 52 and balloon wall 53, which in a connection zone 54 is coupled to the catheter by use of an adhesive 55.

An irrigation channel 56 which opens out via the opening 57 allows the balloon catheter to be used in an anal irrigation kit (not shown). When performing anal irrigation he catheter is inserted into the anal canal of a user. When inserted the balloon wall is radially expanded by injecting gas or liquid through an inflation channel 63 and into the inflation lumen 63 defined between the balloon wall 53 and the outer surface of the catheter body and the first connection zone 54 and a second connection zone (not shown) provided distally of the of the first connection zone. The catheter is thereby retained as the balloon wall is pressed against the wall of the anal canal the balloon catheter. Irrigation fluid is then injected into the bowels through the irrigation channel 56 via the opening 57.

An annular rim 58 is provided in the coupling zone 54. The annular rim defines a planar adhesion surface 59. By planar it should be understood that the surface is mainly flat, i.e. not intentionally curving or uneven. This allows the adhesive 55 to be applied in an even layer on the adhesion surface.

The planar adhesion surface 59 continues, in a direction towards the second connection zone, via transition edge 60, into an outflow surface 61.

The transition edge may be very sharp. For example when using mold injection the tools may define this transition edge and provide and edge having a radius of 0.1 mm, or even less. However, depending on the material used for the balloon wall transition edges having a sharp edge may stress or cut into the material thereby risking that a rupture occurs. Thus, in some embodiment a transition edge having a radius above 0.1, e.g. in the range of 0.5-5 mm or even more may be provided.

In FIG. 2 the outflow surface 61 form an angle α of 90° to the adhesion surface 59. By providing a well-defined change in the contour of the adhesion surface an outflow path is provided, allowing for excess adhesive to be transported away from the balloon wall along the outflow surface 61, thereby minimizing unwanted adhesion resulting in uneven inflation of the balloon. The change in contour also separates the balloon wall from the outer surface of the catheter body thereby preventing adhesion in case adhesive unintentionally is disposed on the inner surface of the balloon wall or on the outer surface of the catheter body.

In order to provide a flush assembly the balloon wall may be countersunk into the catheter body. Providing a recess 62 for receiving the balloon wall may provide such countersink. The depth of the recess substantially corresponds to the thickness of the balloon wall.

From the above and through tests and experience, the following is some of the factors which may influence the inflated shape of the balloon.

When looking at the balloon catheter 51 with reference to the longitudinal axis A-A it should be understood that the balloon wall 53 is a tubular member arranged around the catheter body 52.

The inflation lumen 63 is defined by the balloon wall 53; the outflow surface 61 of the shown coupling zone 54; a corresponding opposite, when seen along the longitudinal axis, outflow surface (not shown) of an opposite not shown coupling zone; and an inflation surface 65 of the catheter body.

The outflow surfaces are arranged annularly around the longitudinal axis, extending perpendicular from an inner radius r₁ to an intermediate radius r₂ of the catheter body. At the inner radius r₁ the outflow surfaces joins the inflations surface 65 at opposite ends thereof. The inflation surface is arranged annularly around the longitudinal axis.

At the intermediate radius r₂ the outflow surfaces joins the respective planar adhesion surfaces, via which the catheter body is attached to the balloon wall as described.

The planar adhesion surface extends along the longitudinal axis from the outflow surface to a recess surface 62. As with the other surfaces described herein, the planar adhesion surface also extends annularly around the longitudinal axis A-A. The recess surface extends radially from the intermediate radius r₂ where it is joined with the planer adhesion surface to an outer radius r₃ where it joins the outer surface 66 of the catheter body 52.

Thus it can be understood that the inflation surface is interposed between two adhesions surfaces 59 (one not shown), where said adhesion surfaces have a radial extent larger than the radial extent of the inflation surface.

In a second embodiment of a balloon catheter 100 as shown in FIG. 3, the planar adhesion surface 101 may form a slight angle β with the longitudinal axis A-A, thereby forming and acute angle with the outflow surface 102 tapering slightly in a direction away from the outflow surface.

The adhesive 105 will thereby primarily move in the tapering direction thereby reducing the amount which is squeezed out onto the outflow surface.

The consideration applied to the radial extent of surfaces above in respect to FIG. 2 can also be applied to the embodiment of FIG. 3. However, it should be noted that the intermediate radius, i.e. the radius from the longitudinal axis to the planer adhesions surface, is not constant since the planar adhesion surface tapers. Accordingly, the radius will decrease from the point where the outflow surface 102 joins the planar adhesion surface 101, where the radius is at its maximum, to where the planar adhesions surface joins a recess surface 103, where the radius is at its minimum.

A third embodiment of a balloon catheter 200 is in section in FIG. 4. The balloon catheter comprises a catheter body 201 to which a tubular balloon wall 203 is joined in a connection zone 204.

An inflation channel 205 allows inflation gas or liquid to be pumped into the inflation lumen 206 in order to inflate the balloon wall radially outwards.

In the connection zone 204 the balloon wall 203 is joined to a first planar adhesive surface 207 and a second planar adhesive surface 208 by an adhesive 209. The two adhesive surfaces are separated by an annular reservoir groove 210. The groove serves to receive excess adhesive coming from the two surfaces. An additional reservoir groove 211 is formed at the distal side of the second adhesive surface and likewise serves to receive excess adhesive.

As in FIG. 2, the first planar adhesive surface forms an angle of 90° with the outflow surface 212.

A balloon catheter as described in respect to FIGS. 2-4 may be adapted to be used for children, where it is important that the balloon wall inflates evenly. Such balloon catheters can have a length (i.e. along the longitudinal axis A-A) of 115 mm and a diameter of 11.5 mm. The balloon wall may have a longitudinal length of 25 mm and may be capable of being inflated to a diameter of at least 70 mm. Of course it can be understood that other embodiments of the balloon catheter may be provided having dimensions making it suitable for adults, or even animals.

The catheter body can be formed of a thermoplastic polymer while the balloon wall can be formed of polychloroprene latex. The adhesive used can be a Medi-Cure 222 gel from Dymax.

The angle between the adhesion surface and the outflow surface is one factor as discussed herein. The sharper the angle, the easier for the adhesive to be transported away and thereby undesirably adhering to balloon wall outside the adhesion surface.

The viscosity of the adhesive is also a factor to consider. If the material has a low viscosity, it will easy run away around the transition edge and onto the outflow surface. However, such viscose adhesives will at the same be more difficult to control and hard to keep on the adhesion surface.

Furthermore, the above embodiments are described in respect to the proximal connection zone. However, as mentioned above a distal connection is also provided and may be provided as a mirror of the features of the proximal connection zone.

The above embodiment as illustrated in FIGS. 2, 3 and 4 are shown with a balloon wall which extends parallel to the longitudinal axis A-A. However, in many case the balloon wall will in its relaxed state have a diameter which is narrower than the catheter. Thus, when applied around the catheter it will due to its elasticity and flexibility follow the contour of the catheter. This will for example result in that in its non-inflated state the inflation volume will be minimal.

Such narrow balloon walls makes it even more difficult to control the distribution of excess joining material, e.g. adhesive, between the balloon wall and the catheter body as the balloon wall creates a ‘splatter’ effect when applied to the catheter, i.e. it randomly displaces the joining material longitudinal away from the joining surfaces.

Thus, it is particularly desirable to control the direction, e.g. via means for guiding, that the joining material is displaced when the balloon wall has a diameter narrower than the catheter. It should be understood that such control may also be desirable in other embodiments where the balloon wall diameter is not narrow, e.g. as the ones described above.

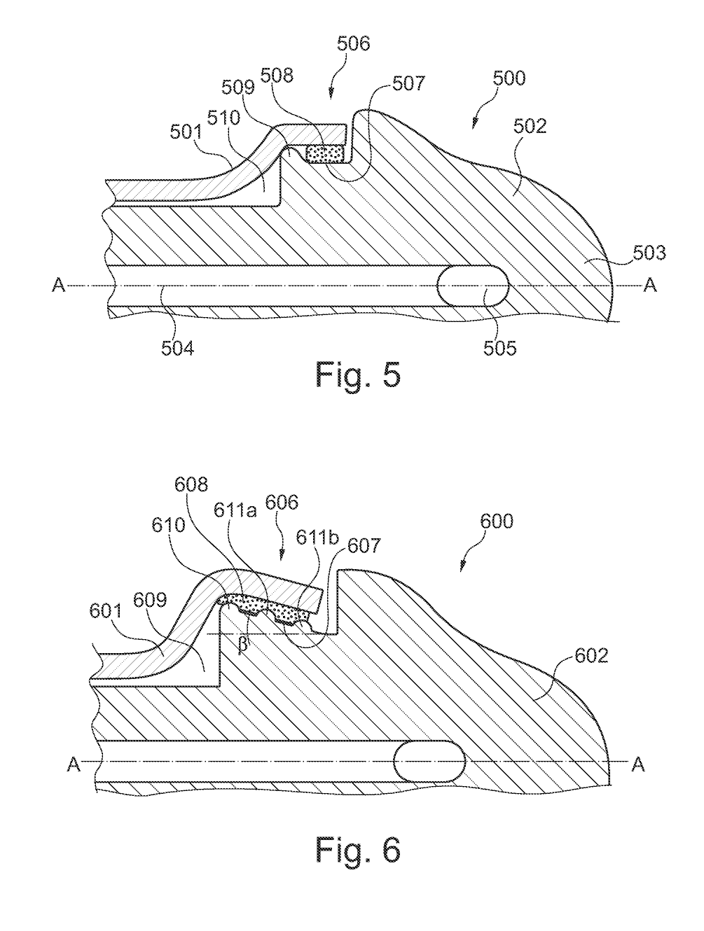

One embodiment of a balloon catheter 500 having a balloon wall of such narrow diameter is shown in FIG. 5.

A cylindrical catheter body 502 is formed with a proximal tip 503 which has a shaped making it suitable for introducing the catheter into the rectal canal of a person. An irrigation channel 504 communicates with irrigation opening 505 so that irrigation fluid pumped through the irrigation channel may be evacuated into the anal canal through the irrigation opening 505.

The catheter body 502 is coupled to a balloon wall 501 in a connection zone 506.

The connection zone comprises an annular joining surface 507 provided on the catheter body 502. An adhesive 508 is applied to the joining surface which connects the balloon wall 501 to the joining surface.

The joining surface 507 has an elevated edge 509 separating the joining surface from the inflation lumen 510. Thus, when the balloon wall is applied and the adhesive is displaced the elevated edge will reduce the risk that adhesive spills into the inflation lumen 510.

Another embodiment of a balloon catheter 600 is illustrated in FIG. 6.

A balloon wall 601 is attached to a catheter body 602 in a connection zone 606. In the connection zone 606 there is provided a joining surface 607 which is connected to the balloon wall 601 via an adhesive 608.

The joining surface 607 tapers inwards in a direction away from the inflation lumen 609 defined by the balloon wall and the catheter body. Accordingly, similar to the embodiment described with respect to FIG. 3 above, the joining surface forms an angle β with the longitudinal axis A-A of the balloon catheter which result in that excess adhesive will be guided in a direction away from the inflation lumen 609.

Similar to the embodiment described with respect to FIG. 5 above the joining surface 607 has an elevated edge 610 separating the joining surface from the inflation lumen 609. Thus, when the balloon wall is applied and the adhesive is displaced the elevated edge will reduce the risk that adhesive spills into the inflation lumen 609.

As can be understood both the tapering of the joining surface and the elevated edge 610 functions as means to guide the adhesive away, so that it does not runs into the inflation lumen. However, depending on the viscosity of the adhesive the guiding means may be too effective. Thus, there is provided two small ribs 611a and 611b which functions as small dams slowing and also retaining some adhesive.

1. A balloon catheter comprising an elongated catheter body extending along a longitudinal axis, where a balloon wall is coupled to the catheter body in at least two annular coupling zones, wherein in at least one of the coupling zones the balloon wall is joined to the catheter body by a joining material, said joining material being provided on a first joining surface of a rim arranged annularly, with a first radius, around the longitudinal axis and extending along the longitudinal axis; and where the joining surface, in a direction towards the opposite coupling zone, continues into an outflow surface which form an angle to the first joining surface, said outflow surface extending from the first radius toward a decreased radius.

2. A balloon catheter according to claim 1, wherein guiding means for guiding the joining material in a direction away from the opposite coupling zone are provided on at least one of the coupling zones.

3. A balloon catheter according to claim 2, wherein the guiding means comprise that the first joining surface forms an angle with the longitudinal axis whereby the surface tapers inwards in the longitudinal direction away from the opposite coupling zone.

4. A balloon catheter according to claim 2, wherein the guiding means comprises an elevated edge provide on at least one joining surface proximal to the opposite coupling zone.

5. A balloon catheter according to claim 2, wherein the guiding means comprises at least one rib provided on the at least one joining surface.

6. A balloon catheter according to claim 2, wherein a first annular reservoir groove is provided on at least one side of the first joining surface.

7. A balloon catheter according to claim 6, wherein a second joining surface is in the longitudinal direction provided on the opposite side of the first annular reservoir groove relative to the first second joining surface.

8. A balloon catheter according to claim 2, wherein the angle that the outflow surface forms to the first joining surface is above 20°.

9. A balloon catheter according to claim 2, wherein the joining material is an adhesive having a viscosity below 10.000 cP.

10. A balloon catheter according to claim 2, any of the preceding claims, wherein the first joining surface is planar.

11. A balloon catheter according to claim 2, wherein the first joining surface is parallel to the longitudinal axis.

12. A balloon catheter according to claim 2, wherein the catheter body is injection moulded.
